(19) United States
(12) Patent Application Publication
(10) Pub. No.: US 2005/0095603 A1
(43) Pub. Date: May 5, 2005

(54) Universal control for nucleic acid amplification

(75) Inventors: Anupama Mokkapati, Santa Clara, CA (US); Michael Ho, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Cepheid, Sunnyvale, CA (US)

(21) Appl. No.: 10/702,528

(22) Filed: Nov. 5, 2003

(51) Int. Cl. C12Q 1/68; C07H 21/04; C12P 19/34

(52) U.S. Cl. 435/6; 435/91.2; 536/24.3

(57) ABSTRACT

The present invention provides a universal internal control system that can be used in a wide variety of amplification reactions, and compositions and methods for performing amplification reactions of nucleic acids.

### CROSS-REFERENCES TO RELATED APPLICATIONS

Not applicable.

### STATEMENT AS TO RIGHTS TO INVENTIONS MADE UNDER FEDERALLY SPONSORED RESEARCH OR DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK

Not applicable.

### FIELD OF THE INVENTION

This invention relates to internal controls for nucleic acid amplification reactions.

### BACKGROUND OF THE INVENTION

In vitro nucleic acid amplification techniques provide powerful tools for detection and analysis of small amounts of nucleic acids. Amplification schemes can be broadly grouped into two classes based on whether the enzymatic amplification reactions are driven by continuous cycling of the temperature between the denaturation temperature, the primer annealing temperature, and the synthesis temperature (thermocyclic amplification), or whether the temperature is kept constant throughout the enzymatic amplification process (isothermal amplification). The polymerase chain reaction (PCR) is a particularly well known and versatile thermocyclic method for the amplification of a nucleic acids (see e.g., PCR Technology: Principles and Applications for DNA Amplification Erlich, ed., (1992); PCR Protocols: A Guide to Methods and Applications, Innis et al., eds, (1990); R. K. Saiki, et al., Science 230:1350 (1985), and U.S. Pat. No. 4,683,202 to Mullis, et al.).

Despite the unquestioned utility of nucleic acid amplification reactions, artifacts frequently arise, usually due to side reactions such as those that occur as a result of mis-priming or primer dimerization. In addition to complicating and confusing the interpretation of results, these artifactual side reactions can deplete the reaction of dNTPs and primers and outcompete the templates for DNA polymerase. Thus, accurate interpretation of the results of an amplification reaction requires that controls capable of detecting and quantitating both false positive and false negative results be included in the reactions.

Controls for amplification reactions employ two basic design schemes, i.e., positive and negative control reactions can be run in separate reaction tubes, or for greater efficiency and accuracy, internal controls can be run in the same reaction tube as the experimental sample. Indeed, numerous variations on these two themes have been described, but internal controls, if available are usually preferred.

In some cases, internal controls utilize different primers to amplify the target of interest and the control (Matsumara et al.; Jpn. J. Clin. Oncol. (1992) 22:335-341). However, most internally controlled PCRs select internal control sequences which can be amplified by the same primers as the target sequence (see, e.g., WO 93/02215 and WO 92/11273). Where the same primers amplify the control and the analyte sequences, the analyte and control sequences may be distinguished by different fragment lengths (Gilliland et al. Proc. Natl. Acad. Sci. USA 1990, 87:2725-2729 and Ursi et al. APMIS 1992, 100:635-639) or by cleavage of the control with a restriction enzyme (Becker and Hahlbroeck; Nucl. Acid Res. 1989, 17:9437-9446). Alternatively, an internal control may be designed to contain a unique probe-binding region that differentiates the control from the amplified target sequence (Rosenstraus et al. J. Clin. Microbiol. 36(1):191-197 (1998)).

In multiplex PCR, a separate internal control sequence may be matched to each target amplified, or if the templates are closely related, a sequence common to all templates may provide the single positive control for amplification (see, e.g., Kaltenboeck, B., et al. J. Clin. Microbiol. 30(5):1098-1104 (1992); Way, J., et al. App. Environ. Microbiol. 59(5):1473-1479 (1993); Wilton, S. et al. PCR Methods Appl. 1:269-273 (1992). Alternatively, adapter-mediated multiplex amplification methods permit a single pair of primers to be used for both the control and each of the multiple targets.

Unfortunately, a pervasive difficulty in the use of internal controls for amplification reactions is keeping amplification of the control polynucleotide from interfering with amplification of the target or detection of the product. This can be particularly difficult when the same primers are used to prime the analyte and control sequences or when the primers used to amplify the control show sequence similarity with regions of the analyte sequence or other nucleic acids in the assay mixture. A further difficulty is that it is generally required that controls be designed specifically for each reaction. Therefore, especially in the case of high throughput diagnostic assays, experimental design and assay efficiency is complicated by the need to design new and different controls for every reaction.

Clearly, there is a need in the art for an effective internal control system for nucleic acid amplification reactions that could be used universally. The ideal control would be uniquely identifiable, and would not interfere with the reaction through mis-priming or competition for reagents. A truly universal internal control would not be substantially similar to any nucleic acid sequences found in nature. Indeed, a universal control would not contain sequences such as those that might be found in a diagnostic laboratory setting, including human, pathogenic organism, normal flora organisms, or environmental organisms. The invention disclosed herein addresses these and other needs.

### SUMMARY OF THE INVENTION

In one aspect, the invention provides an internal control system for monitoring the efficiency of a nucleic acid amplification reaction. The invention comprises a length of non-natural nucleotide sequence comprised of a first gene fragment and a second gene fragment that are linked at a junction defined by a covalent bond between the fragments, wherein the sequences of the first gene fragment and the second gene fragment share less that 50% sequence identity within 100 nucleotides of the junction. The internal control system further comprises a first control primer, that comprises a length of nucleotide sequence that specifically hybridizes at a first melting temperature, at a site across the junction between the first gene fragment and the second gene fragment. This first control primer is able to prime nucleic acid synthesis of the non-natural nucleotide sequence.

In one embodiment, the first gene fragment of the non-natural nucleotide sequence and the second gene fragment of the non-natural nucleotide sequence are each unique sequences derived from organisms of different taxa. In another embodiment, the first gene fragment is derived from Yersinia enterocolitica and the second gene fragment is derived from Tritrichomonas foetus.

In a related embodiment, the non-natural nucleotide sequence further comprises a third gene fragment adjacent to the second gene fragment, and the second and third gene fragments are linked at a junction defined by a covalent bond between the second and third fragments, and a second control primer that comprises a second length of nucleotide sequence that specifically hybridizes at a site across the junction between the second and third gene fragments at a second melting temperature that is within 5° C. of the first melting temperature, wherein the second control primer is able to prime nucleic acid synthesis of the non-natural nucleotide sequence. In one embodiment, the sequences of the second gene fragment and the third gene fragment share less that 50% sequence identity within 100 nucleotides of the junction. In another embodiment, the second gene fragment and the third gene fragment are each unique sequences derived from organisms of different taxa. In a related embodiment, the third gene fragment is derived from a prokaryotic organism, and the second gene fragment is derived from a eukaryotic organism. In other embodiments the first gene fragment and the third gene fragment are unique sequences derived from the same organism, and in a related embodiment are derived from Yersinia enterocolitica. In further embodiments, the internal control system for monitoring the efficiency of a nucleic acid amplification reaction further comprises at least one probe for hybridizing to the second gene fragment.

In one aspect the invention provides a method of performing an amplification reaction, the method comprising the steps of (a) combining in an aqueous solution an internal control comprising a length of a non-natural nucleotide sequence comprised of a first gene fragment and a second gene fragment, linked at a junction defined by a covalent bond between the first and second gene fragments, wherein the sequences of the first and second gene fragments share less that 50% sequence identity within 100 nucleotides of the junction; and a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence; and nucleotides, enzymes, and cofactors necessary to produce an amplification reaction; and (b) amplifying the non-natural nucleotide sequence and amplifying an analyte specific sequence if the analyte specific sequence is present in the solution. In one embodiment the method further comprises the step of detecting the presence or absence of nucleic acid amplification products produced by amplifying the non-natural nucleotide sequence and the analyte specific sequence if the analyte specific sequence is present in the solution. In further embodiments the method also comprises the steps of: (iv) identifying analyte specific and internal control specific amplification products; and (v) comparing the analyte specific and internal control specific amplification products. In some embodiments, the comparison of the analyte specific and internal control specific products is conducted by quantitating the products using real-time analysis. In one embodiment, the detection of the amplification products is conducted by measuring fluorescence. In another embodiment, the non-natural nucleotide sequence and the analyte specific sequence, if present, are amplified by a thermocyclic amplification reaction. In other embodiments, the non-natural nucleotide sequence and the analyte specific sequence, if present, are amplified by an isothermic amplification reaction.

### BRIEF DESCRIPTION OF THE DRAWINGS

FIG. 1 is a graph plotting the cycle threshold (C(t)), and end point fluorescence (EP) achieved in PCR reactions using a universal internal control system of the invention. PCR was carried out under real time conditions using probes designed to hybridize to the control template at 65° C. The graph also depicts the relationship of cycle threshold to end point fluorescence at different starting concentrations of control template.

FIG. 2 is a graph plotting the cycle threshold (C(t)), and end point fluorescence (EP) achieved in PCR reactions using a universal internal control system of the invention. PCR was carried out under real time conditions using probes designed to hybridize to the control template at 56° C. The graph also depicts the relationship of cycle threshold to end point fluorescence at different starting concentrations of control template.

FIG. 3 is a graph plotting end point fluorescence (EP) achieved in multiplex PCR reactions using four different template DNAs. The Figure illustrates the increase in end point fluorescence that accompanies increases in input DNA concentration. The Figure also shows that when concentrations of enzyme and/or other reagents become limiting due to the amplification of many starting molecules, the internal control does not out compete the target template for the limited resource.

### DEFINITIONS

“Covalent bond” as used herein takes it customary meaning, and refers to the bond formed by the sharing of two or more electrons between two atoms. The atoms linked by the covalent bond may be part of a larger molecule such as a sugar molecule or a phosphate group. To say that two gene fragments are “linked at a junction defined by a covalent bond” means that at a reactive, chemically defined location, a reaction has taken place so as to create one, chemically joined, molecule where prior to the reaction, there were two independent molecules. By way of example, but not limitation, the two molecules may gene fragments that are linked through a phosphodiester bond. In this case, the 3′-hydroxl of one sugar moiety of a first nucleotide is joined covalently through a phosphate group to the 5′-hydroxyl group of the sugar moiety of a second, adjacent nucleotide. The reaction to form the bond takes place between the oxygen atom of the 3′-hydroxyl group of the first nucleotide and the phosphorus atom of the phosphate group that is directly linked to the 5′-hydroxyl of the second, adjacent nucleotide. The covalent bonds through which the two gene fragments are linked may also include phosphothioester bonds, or any other appropriate bond that covalently links the fragments such that an amplification primer can hybridize across the junction and prime synthesis of the non-natural nucleotide sequence.

The term “non-natural nucleotide sequence” refers to a nucleotide sequence that does not ordinarily exist in nature. Although fragments or segments of a non-natural nucleotide sequence may show sequence identity with nucleotide sequences ordinarily found in nature, the whole of the non-natural nucleotide sequence, especially the region comprising the 100 nucleotides on either side of the junction between fragments comprising the non-natural nucleotide sequence, is not a sequence that occurs naturally.

“Gene fragment” as used herein refers to any fragment of a gene. Thus, “gene fragment” refers to nucleic acid segments that include coding regions, non-coding regions, and mixtures of coding and non-coding regions.

An “analyte” means a substance whose presence, concentration or amount in a sample is being determined in an assay. An analyte is sometimes referred to as a “target substance” or a “target molecule” or a “target analyte” of an assay. An analyte may also be referred to more specifically. For an analyte that is a nucleic acid, for example, the analyte may be referred to as a “an analyte nucleic acid sequence” or a “target polynucleotide” or a “target sequence” or a “target oligonucleotide,” depending on the particular case. With assays according to the present invention, the analyte is usually a biopolymer or a segment of a biopolymer, but it is not intended that the invention be limited to any specific analyte. Indeed, “analyte nucleic acid sequence” as used herein, refers to any target nucleic acid other than the internal control, whose amplification, by the methods of the invention, is of interest to one of skill in the art.

“Percent (%) nucleic acid sequence identity” is defined as the percentage of nucleotide residues in a particular nucleic acid sequence that are identical between that nucleic acid sequence and one or more nucleic acid sequences with which the particular nucleic acid sequence is being compared. Detailed methods for determining sequence identity can be found in later sections of the disclosure.

“Melting temperature” as used herein refers to the temperature at which a nucleic acid probe will dissociate from its target nucleic acid sequence. The melting temperatures of oligonucleotides are most accurately calculated using nearest neighbor thermodynamic calculations with the formula:
Tₘ primer=ΔH [ΔS+R ln(c/4)]−273.15° C.+16.6 log 10[K+]
where Tₘ is the melting temperature of the oligonucleotide, H is the enthalpy, S is the entropy for helix formation, R is the molar gas constant and c is the concentration of primer/oligonucleotide. Making this calculation for a particular application is most easily accomplished using any of a number of primer design software packages on the market.

In the absence of computer software, those of skill in the art will recognize that a good working approximation of Tₘ (generally valid for oligonucleotides in the 18-24 base range) can be calculated using the formula:
Tₘ=2(A+T)+4(G+C).

“Taxon” as used herein refers to the general term for any taxonomic category such as species, genus, family, order, or phylum.

“Cofactors” as used herein refer to the assorted agents that are sometimes added to an amplification reaction to achieve the desired results. By way of example, but not limitation, such agents can include dimethylsulfoxide (DMSO) or dithiothreotol (DTT). Other agents such as gelatin, bovine serum albumin, and non-ionic detergents (e.g. Tween-20) are also commonly added to amplification reactions (see, e.g. Innis et al. supra). In addition, components of the reaction such as salt, or magnesium may be considered “cofactors” as well. Concentrations of cofactors in any given reaction can be adjusted in accordance with guidance well known in the art, e.g., Innis et al., supra.

“Internal control” as used herein refers to a control reaction run in parallel, in the same container as a reaction of interest, that functions as a standard of comparison.

A “nucleic acid amplification reaction” refers to any chemical, including enzymatic, reaction that results in increased copies of a template nucleic acid sequence. Amplification reactions include, but are not limited to polymerase chain reaction (PCR) and ligase chain reaction (LCR) (see e.g. U.S. Pat. Nos. 4,683,195 and 4,683,202; and, PCR Protocols: A Guide to Methods and Applications, Innis et al., eds, (1990)), strand displacement amplification (SDA, Walker, et al. Nucleic Acids Res. 20(7): 1691-6 (1992); Walker PCR Methods Appl 3(1):1-6 (1993)), transcription-mediated amplification (TMA, Phyffer, et al., J. Clin. Microbiol. 34:834-841 (1996); Yuorinen, et al., J. Clin. Microbiol. 33:1856-1859 (1995)), nucleic acid sequence-based amplification (NASBA, Compton, Nature 350(6313):91-2 (1991), rolling circle amplification (RCA, Lisby, Mol. Biotechnol. 12(1):75-99 (1999)); Hatch et al., Genet. Anal. 15(2):35-40 (1999)) and branched DNA signal amplification (bDNA, Iqbal et al., Mol. Cell Probes 13(4):315-320 (1999)).

A “thermocyclic amplification reaction” refers to the amplification of DNA fragments by subjecting a reaction mixture comprising primer oligonucleotides and a thermostable enzyme to a thermocyclic process that typically comprises either two or three step heating and cooling cycles. The heating and cooling cycles govern the denaturation, and hybridization/elongation steps of the reaction, and are repeated until the amplification is sufficient for the desired application. Two step cycles have a denaturation step followed by a hybridization/elongation step. Three step cycles comprise a denaturation step followed by a hybridization step followed by a separate elongation step. The reactions are preferably carried out in a thermocycler to facilitate incubation at the desired temperatures for the desired period of time. Thermocyclic reactions such as the polymerase chain reaction (PCR) and the ligase chain reaction (LCR) are well known, and are discussed more fully below.

### DETAILED DESCRIPTION OF THE INVENTION

Introduction

The invention provides an internal control system for monitoring the integrity of amplification reagents, inhibition of the reaction from the sample matrix and the efficiency of a nucleic acid amplification reaction. The internal control system comprises a length of non-natural nucleotide sequence comprising a first gene fragment and a second gene fragment, which are linked at a junction defined by a covalent bond. The sequences of the first gene fragment and the second gene fragment share less that 50% sequence identity within 100 nucleotides of the junction. The system further comprises a first control primer of 12-30 nucleotides that specifically hybridizes at a first melting temperature at a site across the junction between the first gene fragment and the second gene fragment. The first control primer is able to prime nucleic acid synthesis of the non-natural nucleotide sequence.

Joining of the two unrelated gene fragments results in a non-natural nucleotide sequence that is unlikely to be found in nature. In particular, the sequences at and around the junction region are exceptionally unique and therefore provide an ideal site at which to direct the design of amplification primers. Thus, the invention provides a universal control system for nucleic acid amplification.

Determining Percent Identity Between Sequences

To practice the methods of the invention, one of skill first needs to determine the percent sequence identity between the sequences chosen to comprise the internal control nucleic acid template sequence. While any method known in the art for making such determinations may be used, for the purpose of the present invention, the BLAST algorithm, described in Altschul et al., J. Mol. Biol. 215, 403-410, (1990) and Karlin et al., PNAS USA 90:5873-5787 (1993) is used for determining sequence identity according to the methods of the invention. A particularly useful BLAST program is the WU-BLAST-2 program (Altschul et al., Methods in Enzymology, 266: 460-480 (1996)). WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent nucleic acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored). Thus, according to the methods of the invention “50% sequence identity” refers to two or more sequences wherein the percentage of identical nucleotide residues between the sequences is 50%.

Designing Primers for Amplification of Internal Control Nucleic Acid Template Sequence

The principles of primer design are well known to those of skill in the art, and are described in a number of references, e.g., Ausubel et al., supra; and PCR Protocols: A Guide to Methods and Applications, Innis et al., eds., 1990, Rychlik, W., Selection of Primers for Polymerase Chain Reaction in B A White, ed., Methods in Molecular Biology, Vol. 15: PCR Protocols: Current Methods and Applications, (1993), pp 31-40, Humana Press, Totowa N.J., and Rychlik et al., Nucleic Acids Research, 18, (12): 6409-6412, and Breslauer et al., Proc. Natl. Acad. Sci. USA, 83: 3746-3750, each of which is herein incorporated by reference. Special primer design considerations for specific non-PCR amplification reactions can also be found, for example, in the following references: strand displacement amplification (SDA) Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995), nucleic acid sequence-based amplification (NASBA) Compton, Nature 350 (6313):91-2 (1991), rolling circle amplification (RCA) Lisby, Mol. Biotechnol. 12(1):75-99 (1999); Hatch et al., Genet. Anal. 15(2):35-40 (1999) and branched DNA signal amplification (bDNA) Iqbal et al., Mol. Cell Probes 13(4):315-320 (1999).

In general, primers that have melting temperatures in the range of 50° C. to about 75° C. are preferred. As is practiced by those skilled in the art, the formula Tₘ=[2(A+T)]+[4(G+C)] can be used to calculate the predicted melting temperature of the primers. Alternatively, commercially available primer design software can be used to more accurately calculate melting temperature, especially when the primers are greater then about 25 nucleotides in length. Primer sequences are frequently selected to have 50-60% G and C composition, which for a 20mer oligonucleotide, implies a melting temperature in the range of 60° C.-68° C. However, the final composition of the primer for the control non-natural nucleic acid sequence will be such that the G-C content allows the control primer to have a melting temperature that matches that of the primer(s) for amplification of the analyte nucleic acid sequence(s).

The flexibility and utility of the universal control system of the invention is facilitated by careful primer design. Adjustments in the melting temperature of the primers permit the development of primers that can bind across the junction of the control non-natural nucleotide sequence at a melting temperature matched to assays for any given analyte sequence. For example, if an amplification assay for a particular analyte sequence or set of analyte sequences requires primers with a melting temperature of 65° C. and an an internal control, the primers that amplify the internal control can be designed so as to have a melting temperature of 65° C.

Melting temperature of the control primer(s) can be adjusted by changing the length of the primer. The primer can therefore be a variety of lengths, and often primers are between 5-50 nucleotides in length, more preferably 10-35 nucleotides in length and most preferably 12-30 nucleotides in length. According to the methods of the invention, the length of the primer will depend on, among other things, the length and melting temperature of the primer(s) for amplification of the analyte nucleic acid sequence(s). Melting temperature of the control primer(s) can also be adjusted by changing the specific binding location of the primers across the junction.

The oligonucleotide primers of the invention may be conveniently synthesized on an automated DNA synthesizer, e.g., an Applied Biosystems, Inc. (Foster City, Calif.) model 392 or 394 DNA/RNA Synthesizer, using standard chemistries, such as phosphoramidite chemistry, e.g., disclosed in the following references: Beaucage and Lyer, Tetrahedron, 48: 2223-2311 (1992); Molko et al., U.S. Pat. No. 4,980,460; Koster et al., U.S. Pat. No. 4,725,677; Caruthers et al., U.S. Pat. Nos. 4,415,732; 4,458,066; and 4,973,679; and the like. Alternative chemistries, e.g., resulting in non-natural backbone groups, such as phosphorothioate, phosphoramidate, and the like, may also be employed provided that the hybridization efficiencies of the resulting oligonucleotides and/or cleavage efficiency of the 5′ to 3′ nuclease activity of the polymerase employed are not adversely affected. The primers can be labeled with radioisotopes, chemiluminescent moieties, or fluorescent moieties.

Methods of Constructing an Internal Control for Nucleic Acid Amplification Reactions

Once the sequences of the gene fragments have been selected and the primers designed, the internal control system of the invention may be constructed using any standard recombinant DNA and molecular cloning techniques. Such techniques are well known in the art and are described more fully in Sambrook et al., Molecular Cloning: A Laboratory Manual; Cold Spring Harbor Laboratory Press, Cold Spring Harbor, 3^{rd} edition (2001), and Ausubel, F. M. et al., Current Protocols in Molecular Biology (1994-1998) John Wiley and Sons, Inc., each of which is herein incorporated by reference.

Sequences for construction of the non-natural control template nucleic acid sequence can be obtained by any method known in the art. For example, PCR can be used to obtain the desired sequence in a variety of ways including, but not limited to; as a subclone from a plasmid, from a cDNA library, or from a composition of isolated genomic sequences. Alternatively, sequences can be obtained by chemical synthesis using an automated DNA synthesizer as described above, or as subclones from restriction digestion of plasmids.

Once obtained, fragments can be joined together by any methods known in the art (Sambrook et al. supra and Ausubel et al. supra). For example, sequences can be joined with DNA ligase, or with PCR. Synthetic linkers may be added to the molecules to be joined, or the molecules may be enzymatically processed before ligation. The joined fragments may be subsequently subcloned into a plasmid or cosmid vector.

Nucleic Acid Amplification Reactions

The internal control system of the invention can be used in any amplification reaction. Amplification reactions take many forms, depending on the nature of the molecule being amplified and on the context in which it occurs. For example amplification reactions may comprise reactions such as polymerase chain reaction (PCR, U.S. Pat. Nos. 4,683,195; 4,683,202; and 4,965,188), nucleic acid sequence based amplification (NASBA, U.S. Pat. Nos. 5,409,818; 5,130,238; and 5,554,517), transcription-mediated amplification (TMA, U.S. Pat. No. 5,437,990), self-sustained sequence replication (3SR, Fahy, et al., PCR Methods & Appl. 1: 25-33, 1991), ligation chain reaction (LCR, U.S. Pat. Nos. 5,494,810 and 5,830,711), continuous amplification reaction or (CAR, U.S. Pat. No. 6,027,897), linked linear amplification of nucleic acids (LLA, U.S. Pat. No. 6,027,923) and strand displacement amplification (SDA, U.S. Pat. Nos. 5,455,166; 5,712,124; 5,648,211; 5,631,147), and methods to increase a signal produced in the presence of a polynucleotide, such as rolling circle amplification (RCA, U.S. Pat. No. 5,854,033), cycling probe reaction (CPR, U.S. Pat. Nos. 4,876,187 and 5,011,769 and 5,660,988), branched chain amplification (U.S. Pat. Nos. 4,775,619 and 5,118,605 and 5,380,833 and 5,629,153) among others. This multitude of methods may be conveniently divided two groups depending on whether the temperature during the reaction is cycled between heating and cooling steps (thermocyclic reactions), or maintained at a constant temperature (isothermic reactions).

Thermocyclic Amplification Reactions

Amplification of an RNA or DNA template using thermocyclic reactions is well known (see e.g. U.S. Pat. Nos. 4,683,195 and 4,683,202; PCR Protocols: A Guide to Methods and Applications Innis et al., eds, 1990, each of which is herein incorporated by reference). Methods such as polymerase chain reaction (PCR) can be used to amplify nucleic acid sequences of target DNA sequences directly from mRNA, from cDNA, from genomic libraries or cDNA libraries. Exemplary PCR reaction conditions typically comprise either two or three step cycles, wherein two step cycles have a denaturation step followed by a hybridization/elongation step, and three step cycles comprise a denaturation step followed by a hybridization step followed by a separate elongation step.

Thermocyclic nucleic acid amplification technologies such as polymerase chain reaction (PCR), and ligase chain reaction (LCR) are well known.

Isothermic Amplification Reactions

Isothermic amplification reactions are also known and can be used according to the methods of the invention. Examples of isothermic amplification reactions include strand displacement amplification (SDA) (Walker, et al. Nucleic Acids Res. 20(7):1691-6 (1992); Walker PCR Methods Appl 3(1):1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350(6313):91-2 (1991), rolling circle amplification (RCA) (Lisby, Mol. Biotechnol. 12(1):75-99 (1999)); Hatch et al., Genet. Anal. 15(2):35-40 (1999)) and branched DNA signal amplification (bDNA) (see, e.g., Iqbal et al., Mol. Cell Probes 13(4):315-320 (1999)). Other amplification methods known to those of skill in the art include CPR (Cycling Probe Reaction), SSR (Self-Sustained Sequence Replication), SDA (Strand Displacement Amplification), QBR (Q-Beta Replicase), Re-AMP (formerly RAMP), RCR (Repair Chain Reaction), TAS (Transcription Based Amplification System), and HCS.

Multiplex Reactions

The methods of the invention can be used in traditional multiplex reactions. Multiplex PCR results in the amplification of multiple polynucleotide fragments in the same reaction (see, e.g., PCR PRIMER, A LABORATORY MANUAL, Dieffenbach, ed. 1995 Cold Spring Harbor Press, pages 157-171, which is herein incorporated by reference). In multiplex PCR, multiple, different target templates can be added and amplified in parallel in the same reaction vessel. Multiplex PCR assays are well known in the art. For example, U.S. Pat. No. 5,582,989 discloses the simultaneous detection of multiple known DNA sequence deletions.

Real-Time Reporters for Multiplex PCR

The universal internal control system provided by the invention may be used in the execution of real time PCR, or “TaqMan” assays. Real time PCR is known in the art. In this embodiment, the universal control system also comprises a probe that binds to the second gene fragment of the non-natural control template. As is known in the art, TaqMan probes contain two dyes, a reporter dye (e.g. 6-FAM) at the 5′ end and a quencher dye (e.g. Black Hole Quencher) at the 3′ end. During the reaction, the 5′ to 3′ nucleolytic activity of the Taq polymerase enzyme cleaves the probe between the reporter and the quencher thus resulting in increased fluorescence of the reporter. Accumulation of PCR products is detected directly by monitoring the increase in fluorescence of the reporter dye.

Quantitation of Amplification Reactions

Accumulation of amplified product can be quantified by any method known to those in the art. For instance, the standard curve method may be used to determine relative or absolute quantitation of amplification products. In other embodiments, amplification reactions can be quantified directly by blotting them onto a solid support and hybridizing with a radioactive nucleic acid probe.

Kits and Solutions of the Invention

The invention also provides kits and solutions for using the universal internal control system of the invention. For example, the invention provides kits that may include one or more reaction vessels that have aliquots of some or all of the universal amplification control system components in them. Aliquots can be in liquid or dried form. The kits can also include written instructions for the use of the kit to amplify and control for amplification of a target sample.

Kits can include, for instance, (1) a universal non-natural control template, and (2) a 5′ control primer and a 3′ control primer. The kit can also include a control probe for real time assays. In addition, the kit can include nucleotides (A, C, G, T) and a DNA polymerase as well as cofactors to facilitate the reaction.

### EXAMPLES

### Example 1

### Construction of an Internal Control System for Nucleic Acid Amplification Comprising a 212 Base Pair Internal Control Template and Amplification Primers

SEQ ID NO:1 illustrates a universal control for nucleic acid amplification reactions designed according to the methods of the invention. Underlined regions on both the ends are derived from Yersinia enterocolitica. The sequence in the middle is derived from Tritrichomonas foetus.

SEQ ID NO:1: Universal internal control comprising sequences from Yersinia enterocolitica and Tritrichomonas foetus.

CAAGCAAGCTTGTGATCCTCCGCC ATTATCCCAAATGGTATAACATTTA GGAC
TTAAAGCTATGCAATTATCACC TTGTTTTTCAACAGCAAGACCTAATATTTTC
TTTTCATCATTAATGCCT TTTGATGGATCAGGCAACCATTTATAAATATGTTC
ATTATAGAATTTATGTA CTTAATGAC ACCAGCCGAAGTCAGTAGTGATTGGG

The individual sequence components from Yersinia enterocolitica and Tritrichomonas foetus comprising SEQ ID NO:1 are first examined for percent sequence identity using the BLAST 2 sequences algorithm for local alignments (Tatiana A. Tatusova, Thomas L. Madden (1999), Blast 2 sequences—a new tool for comparing protein and nucleotide sequences, FEMS Microbiol Lett. 174:247-250). Such a comparison reveals that these individual sequences share no significant sequence homology, thus, they are suitable candidate sequences for construction of a universal internal control for nucleic acid amplification reactions.

Individual sequences can be ligated together by methods well known in the art (Sambrook et al., Molecular Cloning: A Laboratory Manual; Cold Spring Harbor Laboratory Press, Cold Spring Harbor, 3^{rd} edition (2001), and Ausubel, F. M. et al., Current Protocols in Molecular Biology (1994-1998) John Wiley and Sons, Inc.), or alternatively, the entire control template sequence can be synthesized on an automated DNA synthesizer (e.g., an Applied Biosystems, Inc. (Foster City, Calif.) model 392 or 394 DNA/RNA Synthesizer, using standard chemistries, such as phosphoramidite chemistry, e.g., disclosed in the following references: Beaucage and Lyer, Tetrahedron, 48: 2223-2311 (1992); Molko et al., U.S. Pat. No. 4,980,460; Koster et al., U.S. Pat. No. 4,725,677; Caruthers et al., or U.S. Pat. Nos. 4,415,732; 4,458,066; and 4,973,679).

Amplification primers were designed to span the junction of the Y. enterocolitica and T. foetus sequences on each end. A hybridization probe was selected from the T. foetus region in the middle. Primers and hybridization probes were designed to run in amplification reactions at 65° C. and 56° C. assay temperatures in real-time PCR reactions. Primers and probes were designed with ‘Oligo 6’ software from Molecular Biology Insights, Inc., 8685 US Highway 24 West Cascade, Colo. 80809-1333, USA.

Primer and Probe Set for 65° C. Annealing Temperature:

SEQ ID NO:2:
Forward Primer:
5′ TCA CCT TGT TTT ACA GCA AGA C 3′
SEQ ID NO:3:
Reverse Primer:
5′ CTA CTG ACT TCG GCT GGT GTC ATT 3′
SEQ ID NO:4:
Hybridization Probe labeled with CY5:
5′ TGG ATC AGG CAA CCA TTT ATA AAT ATG TTC ATT
AT 3′.

Primer and Probe set for 56° C. annealing temperature:

SEQ ID NO:5:
Forward Primer:
5′ CAT TAT CCC AAA TGG TAT AAC AT 3′
SEQ ID NO:6:
Reverse Primer:
5′ TTC GGC TGG TGT CAT TAA GTA 3′
SEQ ID NO:7:
Hybridization Probe Labeled with TET:
5′ TTA AAG CTA TGC AAT TAT CAC CTT GTT T′ 3.

### Example 2

### Using the Internal Control System in an Amplification Reaction

Limit of Detection Assays:

The internal control functions to monitor the integrity of the PCR reagents and also to monitor inhibition from the sample matrix. To be certain that any negative results obtained from PCR reactions of clinical samples are true negative results, the internal control must give a reliable and detectable signal. Therefore, experiments were conducted to determine the “limit of detection” of the universal internal control system under real-time PCR assay conditions. The primers and probe sets described above in Example 1 were tested at two different temperatures to determine the limit of detection for each primer and probe set, and to demonstrate the efficiency of the system at different temperatures using different protocols.

Probes were labeled with different dyes; the 65° C. probe was labeled with Cy5 and the 56° C. probe was labeled with TET (5-carboxy-tetramethyl-rhodamine). Test reactions, known as simplex assays because they comprise only one template-primer-probe set, were set up for both 56° C. and 65° C. amplification protocols. The limit of detection was determined by serially diluting internal control template DNA over 7 logs concentration, so that the starting concentration of internal control template ranged from 1 copy per 25 μL reaction, to 1 million copies per 25 μL reaction.

For the concentration of starting material to have been at or above the “limit of detection” in a given reaction, a final end point fluorescence of at least 20 must be reached by the end of the protocol. Relative efficiency of a reaction can be determined by comparing the number of amplification cycles required to achieve a particular end point fluorescence.

The reaction conditions and assay protocols for both the simplex experiments are as follows:

For Each 25 μL Reaction:

| | |
|---|---|
| Primers | 200 nM each |
| (Forward and Reverse): | |
| Probe: | 200 nM |
| dNTPs: | 200 μM each |
| MgCl₂: | 6 mM |
| 10× buffer: | 1× |
| Platinum Taq: | 1.25 Units |
| DNA sample: | 1 μL at appropriate dilution |

Assay Protocols:

All the assays were run on Cepheid Smart Cycler, Cepheid Inc., Sunnyvale, Calif.

56° C. Protocol:

Hold: 95° C., 180 s
45 Cycles: 95° C., 5 s; 56° C., 14 s (Optics ON); 72° C., 5 s.
   65° C. Protocol:
Hold: 95° C., 30 s
45 Cycles: 95° C., 1 s; 65° C., 20 s (Optics ON).

For each reaction the cycle threshold (Ct), and the end point fluorescence (EP) were measured. The cycle threshold (Ct), correlates with the log-linear phase of PCR amplification and is the first cycle in which there is significant increase in fluorescence above the background.

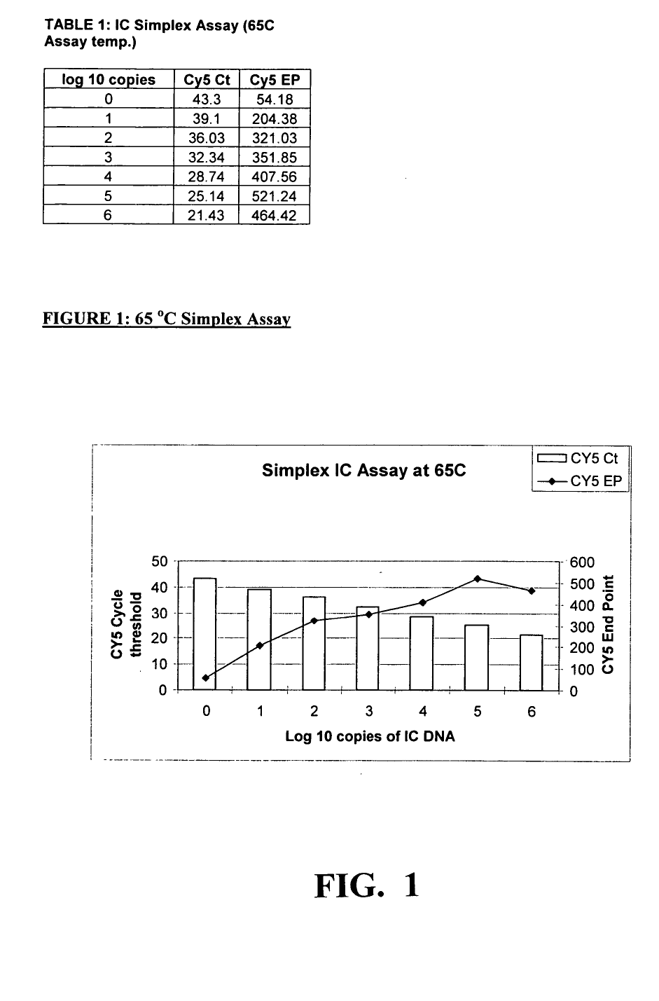

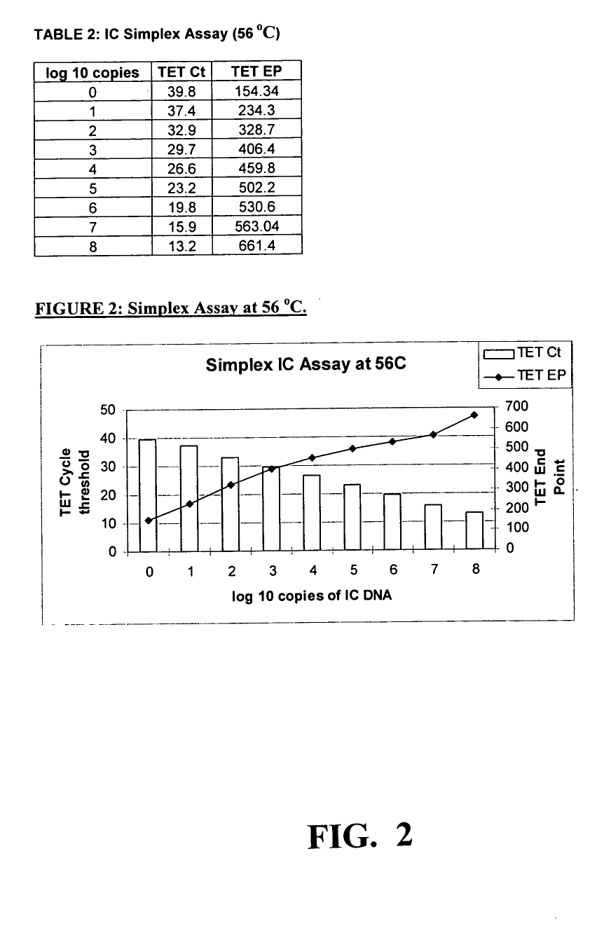

FIGS. 1 and 2 show the results of these limit of detection experiments. For the 65° C. protocol an end point fluorescence of 54 was achieved after 43 amplification cycles when the starting concentration of template DNA was at one copy per reaction. Thus, the limit of detection for this control is one copy per 25 μL reaction. Similarly, the limit of detection for the 56° C. protocol is also one copy per 25 μL reaction.

Comparison of the results shown in FIG. 1, with the results shown in FIG. 2, reveals the relative efficiency of the different amplification protocols. The 56° C. protocol achieves a higher end point fluorescence in fewer cycles than does the 65° C. protocol. Thus, the 56° C. protocol is considered to be more efficient than the 65° C. protocol.

TABLE 1
IC Simplex Assay (65 C.
Assay temp.)

| log 10 copies | Cy5 Ct | Cy5 EP |
|---|---|---|
| 0 | 43.3 | 54.18 |
| 1 | 39.1 | 204.38 |
| 2 | 36.03 | 321.03 |
| 3 | 32.34 | 351.85 |
| 4 | 28.74 | 407.56 |
| 5 | 25.14 | 521.24 |
| 6 | 21.43 | 464.42 |

FIG. 1: 65° C. Simplex Assay

[image]

TABLE 2
IC Simplex Assay (56° C.)

| log 10 copies | TET Ct | TET EP |
|---|---|---|
| 0 | 39.8 | 154.34 |
| 1 | 37.4 | 234.3 |
| 2 | 32.9 | 328.7 |
| 3 | 29.7 | 406.4 |
| 4 | 26.6 | 459.8 |
| 5 | 23.2 | 502.2 |
| 6 | 19.8 | 530.6 |
| 7 | 15.9 | 563.04 |
| 8 | 13.2 | 661.4 |

FIG. 2: Simplex Assay at 56° C.

[image]

Cross Reactivity Assays

A set of assays was carried out to determine whether an internal control constructed according to the methods of the invention would be detected uniquely, or whether the control primers would non-specifically amplify other sequences present in a clinical sample.

The Yersinia enterocolitica and Tritrichomonas foetus sequences comprising the internal control template of SEQ ID NO:1, and the 56° C. primers, i.e. SEQ ID NO:5 and SEQ ID NO:6, were tested for their identity to the sequences of other organisms for which sequence information is available using sequence data from GenBank. Comparisons were made using the BLAST algorithm (Atschul et al. supra). No significant sequence identity was found with any of the sequences tested. Experiments were then carried out with 100 clinical samples to test whether or not just by chance, the 56° C. primers would amplify any of the sequences in any clinical sample.

The experiments were set up as follows. 100 clinical samples were tested in PCR reactions using the 56° C. primers and a FAM-labeled 56° C. probe of Example 1. Probe was added to the 25 μL reactions at a concentration of 300 nM. Internal control primers were at 200 nM each and the remaining reaction components were: dNTPs: 200 μM each, MgCl₂:6 mM, 10× buffer: 1×Platinum Taq: 1.25 Units. Reactions were carried out according to the 56° C. protocol used in the limit of detection assays (,i.e. Hold: 95° C., 180 s; 45 Cycles: 95° C., 5 s; 56° C., 14 s (Optics ON); 72° C., 5 s) in a Cepheid Smart Cycler (Cepheid Inc., Sunnyvale, Calif.).

None of the 100 clinical samples gave any detectable end point fluorescence signal on completion of the 56° C. reaction protocol. Thus, the primers for an internal control template designed according to the methods of the invention uniquely amplify the internal control template DNA.

Fourplex Assays

Further experiments tested the ability of the universal internal control to perform in multiplex PCR reactions involving three or more target templates. A “fourplex” assay was carried out to make this determination. The fourplex assay was developed at Cepheid (Hoffmaster et al. (2002) Emerging Infective Diseases vol. 8:1178-1181).

The fourplex assay involves specific detection of two virulence plasmids from Bacillus anthracis, pXO1 and pXO2, and simultaneous specific detection of two internal controls constructed according to the methods of the invention, UIC and CIC (the internal control of Example 1). Target probes to pXO1 and pXO2, were labeled with FAM (6-carboxy-fluorescein phosphoramidite, pXO1) and CY3 (pXO2) dyes and the internal control probes were labeled with TxRed (UIC) and CY5 (CIC).

Fourplex experiments test the ability of the end point fluorescence signal from the internal controls to be detected regardless of how small the internal control template concentration is relative to the target template concentration. Also, these experiments test whether or not the controls will outcompete the target template when enzyme and/or other reagent concentrations become limiting. For the fourplex assay the concentration of the internal control template DNAs was the same in every reaction. Specifically, the CIC control was kept at 1000 copies per 25 μL reaction whereas the UIC control was used at 280 copies per 25 μL reaction. The DNA of the target plasmids was serially diluted over 6 logs of concentration so that the target was present at concentrations ranging from 0-10,000 copies per 25 μL reaction.

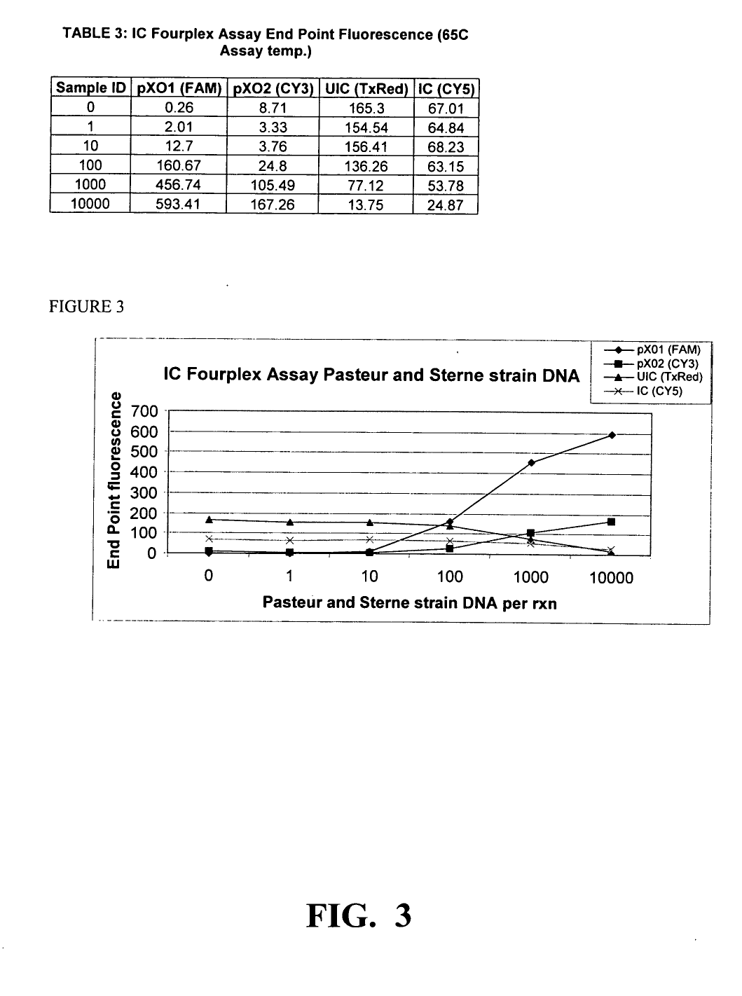

As can be seen in FIG. 3, the endpoint fluorescence of the internal controls is detectable in every reaction. Thus, the internal control is suitable for use with target templates that may vary over a wide range of concentrations. In addition the control does not out compete the target DNA when enzyme concentrations are limiting. This is evident in FIG. 3 wherein the end point fluorescence signal of the internal controls decreases when the starting concentration of target template DNA is high. Thus, internal controls for amplification reactions designed according to the methods of the invention, are effective for use in multiplex amplification reactions.

TABLE 3
IC Fourplex Assay End Point Fluorescence
(65 C. Assay temp.)

| Sample ID | pXO1 (FAM) | pXO2 (CY3) | UIC (TxRed) | IC (CY5) |
|---|---|---|---|---|
| 0 | 0.26 | 8.71 | 165.3 | 67.01 |
| 1 | 2.01 | 3.33 | 154.54 | 64.84 |
| 10 | 12.7 | 3.76 | 156.41 | 68.23 |
| 100 | 160.67 | 24.8 | 136.26 | 63.15 |
| 1000 | 456.74 | 105.49 | 77.12 | 53.78 |
| 10000 | 593.41 | 167.26 | 13.75 | 24.87 |

FIG. 3

[image]

1. An internal control system for monitoring the efficiency of a nucleic acid amplification reaction, the internal control system comprising:
a) a length of a non-natural nucleotide sequence comprising a first gene fragment and a second gene fragment, linked at a junction defined by a covalent bond between the first and second gene fragments, wherein the sequences of the first gene fragment and the second gene fragment share less that 50% sequence identity within 100 nucleotides of the junction; and
b) a first control primer comprising a length of nucleotide sequence that specifically hybridizes at a first melting temperature at a site across the junction between the first and second gene fragments, wherein the first control primer is able to prime nucleic acid synthesis of the control nucleotide sequence.

2. The internal control system of claim 1, wherein the first gene fragment of the non-natural nucleotide sequence and the second gene fragment of the non-natural nucleotide sequence are each unique sequences derived from organisms of different taxa.

3. The internal control system of claim 2, wherein the first gene fragment is derived from a prokaryotic organism, and the second gene fragment is derived from a eukaryotic organism.

4. The internal control system of claim 3, wherein the first gene fragment is derived from Yersinia enterocolitica and the second gene fragment is derived from Tritrichomonas foetus.

5. The internal control system of claim 1, the primer has a length in the range of 5-50 nucleotides.

6. The internal control system of claim 1, the primer has a length in the range of 10-35 nucleotides.

7. The internal control system of claim 1, the primer has a length in the range of 12-30 nucleotides.

8. The internal control system of claim 1, wherein the non-natural nucleotide sequence further comprises a third gene fragment adjacent to the second gene fragment, wherein the second and third gene fragments are linked at a junction defined by a covalent bond between the second and third fragments, and wherein the system further comprises:
a second control primer comprising a second length of nucleotide sequence that specifically hybridizes at a site across the junction between the second and third gene fragments at a second melting temperature that is within 5° C. of the first melting temperature, the second control primer being able to prime nucleic acid synthesis of the non-natural nucleotide sequence.

9. The internal control system of claim 8, wherein the sequences of the second gene fragment and the third gene fragment share less that 50% sequence identity within 100 nucleotides of the junction.

10. The internal control system of claim 8, wherein the second gene fragment and the third gene fragment are each unique sequences derived from organisms of different taxa.

11. The internal control system of claim 10, wherein the third gene fragment is derived from a prokaryotic organism, and the second gene fragment is derived from a eukaryotic organism.

12. The internal control system of claim 8, wherein the first gene fragment and the third gene fragment are unique sequences derived from the same organism.

13. The internal control system of claim 12, wherein the first and third gene fragments are derived from Yersinia enterocolitica.

14. The internal control system of claim 8, wherein the second gene fragment is from a different organism than the first and third gene fragments of the non-natural nucleotide sequence.

15. The internal control system of claim 8, wherein the first and third gene fragments are derived from the bacterium Yersinia enterocolitica, and the second gene fragment is derived from the parasitic eukaryote, Tritrichomonas foetus.

16. The internal control system of claim 15, wherein the first and third gene fragments derived from the bacterium Yersinia enterocolitica are 25 base pair fragments of the Yersinia enterocolitica heat-stable enterotoxin gene, and the second gene fragment derived from the parasitic eukaryote Tritrichomonas foetus is a 162 base pair fragment from an unknown gene of Tritrichomonas foetus.

17. The internal control system of claim 1, further comprising at least one probe for hybridizing to the second gene fragment.

18. The internal control system of claim 8, further comprising at least one probe for hybridizing to the second gene fragment.

19. An internal control system for monitoring the efficiency of a nucleic acid amplification reaction, the internal control system comprising:
a) a length of a non-natural nucleotide sequence comprising a first gene fragment and a second gene fragment, linked at a junction defined by a covalent bond between the first and second gene fragments, wherein the first and second gene fragments are each unique sequences derived from organisms of different taxa; and
b) a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence.

20. The internal control system of claim 19, wherein the sequences of the first gene fragment and the second gene fragment share less that 50% sequence identity within 100 nucleotides of the junction.

21. The internal control system of claim 19, wherein the first gene fragment is derived from a prokaryotic organism, and the second gene fragment is derived from a eukaryotic organism.

22. The internal control system of claim 21, wherein the first gene fragment is derived from Yersinia enterocolitica and the second gene fragment is derived from Tritrichomonas foetus.

23. The internal control system of claim 19, the primer has a length in the range of 5-50 nucleotides.

24. The internal control system of claim 19, the primer has a length in the range of 10-35 nucleotides.

25. The internal control system of claim 19, the primer has a length in the range of 12-30 nucleotides.

26. The internal control system of claim 19, wherein the non-natural nucleotide sequence further comprises a third gene fragment adjacent to the second gene fragment, wherein the second and third gene fragments are linked at a junction defined by a covalent bond between the second and third fragments, and wherein the system further comprises:
a second control primer comprising a second length of nucleotide sequence that specifically hybridizes at a site across the junction between the second and third gene fragments at a second melting temperature that is within 5° C. of the first melting temperature, the second control primer being able to prime nucleic acid synthesis of the non-natural nucleotide sequence.

27. The internal control system of claim 26, wherein the sequences of the second gene fragment and the third gene fragment share less that 50% sequence identity within 100 nucleotides of the junction.

28. The internal control system of claim 26, wherein the second gene fragment and the third gene fragment are each unique sequences derived from organisms of different taxa.

29. The internal control system of claim 28, wherein the third gene fragment is derived from a prokaryotic organism, and the second gene fragment is derived from a eukaryotic organism.

30. The internal control system of claim 26, wherein the first gene fragment and the third gene fragment are unique sequences derived from the same organism.

31. The internal control system of claim 30, wherein the first and third gene fragments are derived from Yersinia enterocolitica.

32. The internal control system of claim 26, wherein the second gene fragment is from a different organism than the first and third gene fragments of the non-natural nucleotide sequence.

33. The internal control system of claim 26, wherein the first and third gene fragments are derived from the bacterium Yersinia enterocolitica, and the second gene fragment is derived from the parasitic eukaryote, Tritrichomonas foetus.

34. The internal control system of claim 33, wherein the first and third gene fragments derived from the bacterium Yersinia enterocolitica are 25 base pair fragments of the Yersinia enterocolitica heat-stable enterotoxin gene, and the second gene fragment derived from the parasitic eukaryote Tritrichomonas foetus is a 162 base pair fragment from an unknown gene of Tritrichomonas foetus.

35. The internal control system of claim 19, further comprising at least one probe for hybridizing to the second gene fragment.

36. The internal control system of claim 26, further comprising at least one probe for hybridizing to the second gene fragment.

37. An internal control system for monitoring the efficiency of a nucleic acid amplification reaction, the internal control system comprising:
a) a length of a non-natural nucleotide sequence comprising a first gene fragment and a second gene fragment, linked at a junction defined by a covalent bond between the first and second gene fragments, wherein the first gene fragment is derived from a prokaryotic organism and the second gene fragment is derived from a eukaryotic organism; and
b) a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence.

38. The internal control system of claim 37, wherein the sequences of the first gene fragment and the second gene fragment share less that 50% sequence identity within 100 nucleotides of the junction.

39. The internal control system of claim 37, wherein the first and second gene fragments are each unique sequences derived from organisms of different taxa.

40. The internal control system of claim 37, wherein the first gene fragment is derived from Yersinia enterocolitica and the second gene fragment is derived from Tritrichomonas foetus.

41. The internal control system of claim 37, the primer has a length in the range of 5-50 nucleotides.

42. The internal control system of claim 37, the primer has a length in the range of 10-35 nucleotides.

43. The internal control system of claim 37, the primer has a length in the range of 12-30 nucleotides.

44. The internal control system of claim 37, wherein the non-natural nucleotide sequence further comprises a third gene fragment adjacent to the second gene fragment, wherein the second and third gene fragments are linked at a junction defined by a covalent bond between the second and third fragments, and wherein the system further comprises:
a second control primer comprising a second length of nucleotide sequence that specifically hybridizes at a site across the junction between the second and third gene fragments at a second melting temperature that is within 5° C. of the first melting temperature, the second control primer being able to prime nucleic acid synthesis of the non-natural nucleotide sequence.

45. The internal control system of claim 44, wherein the sequences of the second gene fragment and the third gene fragment share less that 50% sequence identity within 100 nucleotides of the junction.

46. The internal control system of claim 44, wherein the second gene fragment and the third gene fragment are each unique sequences derived from organisms of different taxa.

47. The internal control system of claim 46, wherein the third gene fragment is derived from a prokaryotic organism, and the second gene fragment is derived from a eukaryotic organism.

48. The internal control system of claim 44, wherein the first gene fragment and the third gene fragment are unique sequences derived from the same organism.

49. The internal control system of claim 48, wherein the first and third gene fragments are derived from Yersinia enterocolitica.

50. The internal control system of claim 44, wherein the second gene fragment is from a different organism than the first and third gene fragments of the non-natural nucleotide sequence.

51. The internal control system of claim 44, wherein the first and third gene fragments are derived from the bacterium Yersinia enterocolitica, and the second gene fragment is derived from the parasitic eukaryote, Tritrichomonas foetus.

52. The internal control system of claim 51, wherein the first and third gene fragments derived from the bacterium Yersinia enterocolitica are 25 base pair fragments of the Yersinia enterocolitica heat-stable enterotoxin gene, and the second gene fragment derived from the parasitic eukaryote Tritrichomonas foetus is a 162 base pair fragment from an unknown gene of Tritrichomonas foetus.

53. The internal control system of claim 37, further comprising at least one probe for hybridizing to the second gene fragment.

54. The internal control system of claim 37, further comprising at least one probe for hybridizing to the second gene fragment.

55. A method of performing an amplification reaction, the method comprising the step of:
(a) combining in an aqueous solution:
(i) an internal control comprising a length of a non-natural nucleotide sequence comprising a first gene fragment and a second gene fragment, linked at a junction defined by a covalent bond between the first and second gene fragments, wherein the sequences of the first and second gene fragments share less that 50% sequence identity within 100 nucleotides of the junction;
(ii) a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence; and
(iii) nucleotides, enzymes, and cofactors necessary to produce an amplification reaction; and
(b) amplifying the non-natural nucleotide sequence and amplifying an analyte specific sequence if the analyte specific sequence is present in the solution.

56. The method of claim 55, further comprising the step of detecting the presence or absence of nucleic acid amplification products produced by amplifying the non-natural nucleotide sequence and the analyte specific sequence if the analyte specific sequence is present in the solution.

57. The method of claim 55, further comprising the steps of:
(iv) identifying analyte specific and internal control specific amplification products; and
(v) comparing the analyte specific and internal control specific amplification products.

58. The method of claim 57, wherein the comparison of the analyte specific and internal control specific products is conducted by quantitating the products using real-time analysis.

59. The method of claim 56, wherein the detection of the amplification products is conducted by measuring fluorescence.

60. The method of claim 55, wherein the non-natural nucleotide sequence and the analyte specific sequence, if present, are amplified by a thermocyclic amplification reaction.

61. The method of claim 60, wherein the thermocyclic amplification reaction is a polymerase chain reaction (PCR).

62. The method of claim 55, wherein the non-natural nucleotide sequence and the analyte specific sequence, if present, are amplified by an isothermic amplification reaction.

63. The method of claim 62, wherein the isothermic amplification reaction is transcription-mediated amplification (TMA).

64. A method of performing an amplification reaction, the method comprising the step of:
(a) combining in an aqueous solution:
(i) an internal control comprising a length of a non-natural nucleotide sequence comprising a first gene fragment and a second gene fragment, linked at a junction defined by a covalent bond between the first and second gene fragments, wherein the first and second gene fragments are each unique sequences derived from organisms of different taxa;
(ii) a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence; and
(iii) nucleotides, enzymes, and cofactors necessary to produce an amplification reaction; and
(b) amplifying the non-natural nucleotide sequence and amplifying an analyte specific sequence if the analyte specific sequence is present in the solution.

65. The method of claim 64, further comprising the step of detecting the presence or absence of nucleic acid amplification products produced by amplifying the non-natural nucleotide sequence and the analyte specific sequence if the analyte specific sequence is present in the solution.

66. The method of claim 64, further comprising the steps of:
(iv) identifying analyte specific and internal control specific amplification products; and
(v) comparing the analyte specific and internal control specific amplification products.

67. The method of claim 66, wherein the comparison of the analyte 2 specific and internal control specific products is conducted by quantitating the products using 3 real-time analysis.

68. The method of claim 65, wherein the detection of the amplification 2 products is conducted by measuring fluorescence.

69. The method of claim 64, wherein the non-natural nucleotide sequence 2 and the analyte specific sequence, if present, are amplified by a thermocyclic amplification 3 reaction.

70. The method of claim 69, wherein the thermocyclic amplification 2 reaction is a polymerase chain reaction (PCR).

71. The method of claim 64, wherein the non-natural nucleotide sequence 2 and the analyte specific sequence, if present, are amplified by an isothermic amplification 3 reaction.

72. The method of claim 71, wherein the isothermic amplification reaction is transcription-mediated amplification (TMA).

73. A method of performing an amplification reaction, the method comprising the step of:
(a) combining in an aqueous solution:
(i) an internal control comprising a length of a non-natural nucleotide sequence comprising a first gene fragment and a second gene fragment, linked at a junction defined by a covalent bond between the first and second gene fragments, wherein the first gene fragment is derived from a prokaryotic organism, and the second gene fragment is derived from a eukaryotic organism;
(ii) a first control primer comprising a length of nucleotide sequence that specifically hybridizes at a first melting temperature at a site across the junction between the first and second gene fragments, wherein the first control primer is able to prime nucleic acid synthesis of the non-natural nucleotide sequence; and
(iii) nucleotides, enzymes, and cofactors necessary to produce an amplification reaction; and
(b) amplifying the non-natural nucleotide sequence and amplifying an analyte specific sequence if the analyte specific sequence is present in the solution.

74. The method of claim 73, further comprising the step of detecting the presence or absence of nucleic acid amplification products produced by amplifying the non-natural nucleotide sequence and the analyte specific sequence if the analyte specific sequence is present in the solution.

75. The method of claim 73, further comprising the steps of:
(iv) identifying analyte specific and internal control specific amplification products; and
(v) comparing the analyte specific and internal control specific amplification products.

76. The method of claim 75, wherein the comparison of the analyte specific and internal control specific products is conducted by quantitating the products using real-time analysis.

77. The method of claim 74, wherein the detection of the amplification products is conducted by measuring fluorescence.

78. The method of claim 73, wherein the non-natural nucleotide sequence and the analyte specific sequence, if present, are amplified by a thermocyclic amplification reaction.

79. The method of claim 78, wherein the thermocyclic amplification reaction is a polymerase chain reaction (PCR).

80. The method of claim 73, wherein the non-natural nucleotide sequence and the analyte specific sequence, if present, are amplified by an isothermic amplification reaction.

81. The method of claim 80, wherein the isothermic amplification reaction is transcription-mediated amplification (TMA).


SEQUENCE LISTING (7 sequences)

SEQ ID NO: 1 - DNA, 211 bp, Artificial Sequence
  Description of Artificial Sequenceuniversal internal control template
caagcaagcttgtgatcctccgccattatcccaaatggtataacatttaggacttaaagctatgcaattatcaccttgtttttcaacagcaagacctaatattttcttttcatcattaatgccttttgatggatcaggcaaccatttataaatatgttcattatagaatttatgtacttaatgacaccagccgaagtcagtagtgattggg

SEQ ID NO: 2 - DNA, 22 bp, Artificial Sequence
  Description of Artificial Sequence65 degree C annealing temperature forward primer
tcaccttgttttacagcaagac

SEQ ID NO: 3 - DNA, 24 bp, Artificial Sequence
  Description of Artificial Sequence65 degree C annealing temperature reverse primer
ctactgacttcggctggtgtcatt

SEQ ID NO: 4 - DNA, 35 bp, Artificial Sequence
  Description of Artificial Sequence65 degree C annealing temperature hybridization probe labeled with CY5
tggatcaggcaaccatttataaatatgttcattat

SEQ ID NO: 5 - DNA, 23 bp, Artificial Sequence
  Description of Artificial Sequence56 degree C annealing temperature forward primer
cattatcccaaatggtataacat

SEQ ID NO: 6 - DNA, 21 bp, Artificial Sequence
  Description of Artificial Sequence56 degree C annealing temperature reverse primer
ttcggctggtgtcattaagta

SEQ ID NO: 7 - DNA, 28 bp, Artificial Sequence
  Description of Artificial Sequence56 degree C annealing temperature hybridization probe labeled with 5-carboxy-tetramethyl-rhodamine (TET)
ttaaagctatgcaattatcaccttgttt